(19) United States
(12) Patent Application Publication
(10) Pub. No.: US 2024/0050407 A1
(43) Pub. Date: Feb. 15, 2024

(54) METHODS FOR DELAY OF EJACULATION IN HUMAN MALES

(71) Applicant: Kanna Health Limited, Kent (GB)

(72) Inventor: Ryan PROTZKO, Alameda, CA (US)

(21) Appl. No.: 18/268,950

(22) PCT Filed: Dec. 21, 2021
(86) PCT No.: PCT/US2021/064653

§ 371 Date: Jun. 21, 2023

Related U.S. Application Data
(60) Provisional application No. 63/128,317, filed on Dec. 21, 2020.

(51) Int. Cl. A61K 31/403; A61K 9/00; A61K 36/185; A61P 15/12

(52) U.S. Cl. CPC A61K 31/403; A61K 9/006; A61K 36/185; A61P 15/12

(57) ABSTRACT

The present disclosure provides a method of treating a sexual dysfunction in a human male subject in need thereof comprising administering an effective amount of a mesembrine alkaloid or salt thereof to the subject in need thereof. Also provided are methods of delaying orgasm and/or ejaculation in human male subjects.

### CROSS-REFERENCE TO RELATED APPLICATIONS

This is an international PCT application claiming priority to U.S. Provisional Application No. 63/128,317, filed Dec. 21, 2020, the content of which is incorporated by reference in its entirety.

### FIELD

The present disclosure is related to the field of sexual dysfunction. In particular, the disclosure provides compositions and methods for delaying ejaculation, delaying orgasm and preventing premature ejaculation during sexual activity in human males.

### BACKGROUND

The estimated prevalence of premature ejaculation is about 22-38% of the male population. Unlike male erectile dysfunction, premature ejaculation has no definite correlation with age. Taking an average prevalence of 30%, that would make an estimated 48 million sufferers in the US (males ages 18-65 was 159 million in 2020). Premature ejaculation is defined as persistent or recurrent ejaculation with minimal sexual stimulation before, upon, or shortly after penetration, and before ejaculation is wished to occur by the individual. Such ejaculation that occurs sooner than desired is often disappointing and can lead to other sexual dysfunctions including erectile difficulties, female inorgasmia, low sexual desire, and sexual aversion. Males experiencing premature ejaculation can also develop mood disorders including anxiety and depression. There is a need in the art for therapeutics to treat premature ejaculation in human males.

### SUMMARY

In some embodiments, the present disclosure provides a method of delaying ejaculation in a human male subject comprising administering an effective amount of a mesembrine alkaloid or salt thereof to the subject.

In some embodiments, the present disclosure provides a method of delaying orgasm in a human male subject comprising administering an effective amount of a mesembrine alkaloid or salt thereof to the subject. In some embodiments, the subject suffers from premature ejaculation. In some embodiments, the subject is a healthy male who does not suffer from premature ejaculation.

In some embodiments, the mesembrine alkaloid is selected from mesembrine, mesembrenone, Δ⁷mesembrenone, 4′-O-demethylmesembranol, mesembrenol, 4′-O-demethylmesembrenol, mesembranol, 4′-O-demethylmesembrenone, sceletenone, N-demethly-N-formulmesembrenone, O-acetylmesembrenol, mesembrane, N-demethelymesembrenol, N-demethylmesembranol, hordenine, joubertiamine, dehydrojoubertiamine, dehydrojoubertiamine, joubertinamine, O-methydehydrojoubertiamine, O-methyljoubertiamine, O-methyldihydrojoubertiamine, 3′-methoxy-4′-O-methjoubertiamine, 3′-methoxy-4′-O-methyljoubertiaminol, 4-(3,4-dimethoxyphenyl-4-[2-acetylmethylamino)ethyl]cyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine. In some embodiments, the mesembrine alkaloid is selected from mesembrine, mesembrenone, mesembrenol, and tortuosamine. In sonic embodiments, the mesembrine alkaloid is a compound of Formula (I).

In some embodiments, the mesembrine alkaloid or salt thereof is administered sublingually, nasally, orally, parenterally, buccally, rectally, topically, transdermally, transurethrally, or by intracavernosal injection. In some embodiments, is formulated with one or more pharmaceutically acceptable excipients. In some embodiments, the mesembrine alkaloid or salt thereof is formulated in a dosage form selected from a liquid, a lozenge, a fast disintegrating tablet, a lyophilized preparation, a film, a spray, or a mucoadhesive. In some embodiments, the spray is an oral spray, a nasal spray, or a topical spray.

In some embodiments, the mesembrine alkaloid or salt thereof is administered as a single daily dose. In some embodiments, the mesembrine alkaloid or salt thereof is administered as multiple daily doses. In some embodiments, the mesembrine alkaloid or salt thereof is administered two, three, four, or five times daily. In some embodiments, the mesembrine alkaloid or salt thereof is administered three times daily.

In some embodiments, each dose comprises at least 0.001 mg of the mesembrine alkaloid or salt thereof. In some embodiments, each dose comprises at least 0.01 mg of the mesembrine alkaloid or salt thereof. In some embodiments, each dose comprises between about 0.001 mg and about 500 mg of the mesembrine alkaloid or salt thereof. In some embodiments, each dose comprises between about 500 mg and about 1000 mg of the mesembrine alkaloid or salt thereof

In some embodiments, the mesembrine alkaloid or salt thereof is administered at least 10 minutes, at least 20 minutes, at least 30 minutes, at least one hour, at least 2 hours, at least 3 hours, at least 4 hours, or at least 5 hours prior to sexual activity. In some embodiments, the mesembrine alkaloid or salt thereof is administered about one hour prior to sexual activity. In some embodiments, the mesembrine alkaloid or salt thereof is administered about 20 minutes prior to sexual activity.

In some embodiments, the sexual dysfunction is premature ejaculation. In some embodiments, the administration of the mesembrine alkaloid or salt thereof prevents premature ejaculation. In some embodiments, the administration of the mesembrine alkaloid or salt thereof extends the amount of time during sexual activity before ejaculation occurs. In some embodiments, the method does not result in a concurrent increase or onset of negative symptoms such as low sex drive or difficulty obtaining or maintaining an erection.

In some embodiments, the mesembrine alkaloid is an extract from a botanical source. In some embodiments, the extract is a substantially pure extract of the mesembrine alkaloid. In some embodiments, the mesembrine alkaloid is synthesized.

In some embodiments, the mesembrine alkaloid is administered in combination with another drug product.

### BRIEF DESCRIPTION OF THE FIGURES

FIG. 1 provides structures and identifying information for the main Sceletium alkaloids of current commercial and pharmacological interest. The numbering system is shown for the 3a-phenyl-cis-octahydroindole core that is characteristic of the mesembrine subgroup of Sceletium alkaloids. The average (MW) and monoisotopic (Exact Mass) molecular weights are given along with the molecular formula and Chemical Abstracts Service (CAS) number for each compound.

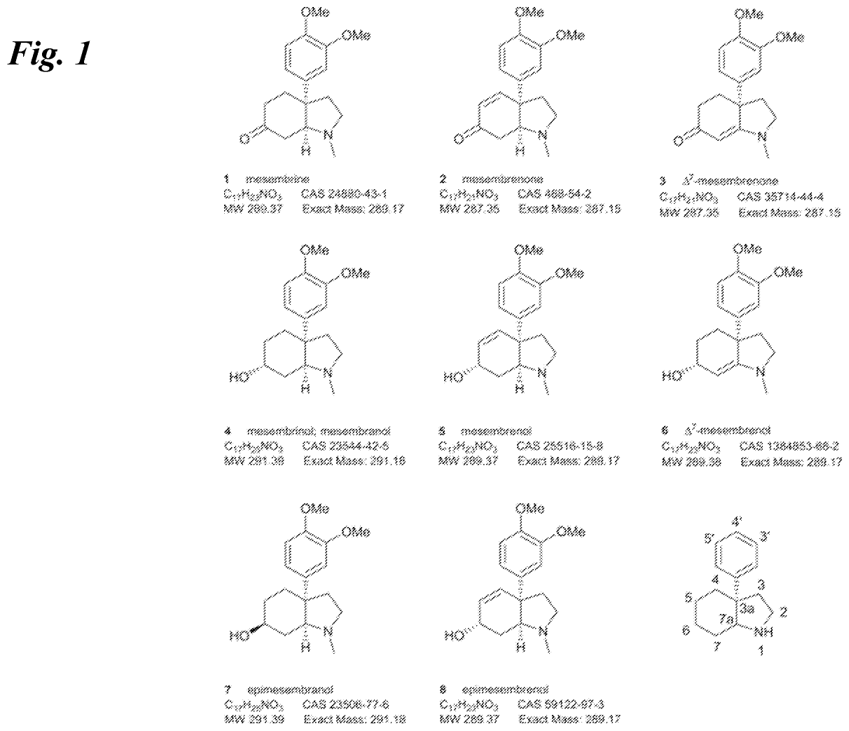

FIG. 2 provides a brief overview of the experimental protocols for PCA-induced ejaculation models in rats.

FIG. 3 shows ejaculation latency data for rats treated with varying doses of mesembrine.

FIG. 4 shows changes in seminal vesicle pressure amplitude in rats treated with varying doses of mesembrine.

FIG. 5 changes in seminal vesicle pressure AUC in rats treated with varying doses of mesembrine

FIG. 6 shows ejaculation latency data for rats treated with mesembrenone.

FIG. 7 shows changes in seminal vesicle pressure amplitude in rats treated with mesembrenone.

FIG. 8 shows changes in seminal vesicle pressure AUC in rats treated with mesembrenone.

FIG. 9 shows ejaculation latency data for rats treated with varying doses of mesembrenol or mesembranol.

FIG. 10 shows changes in seminal vesicle pressure amplitude for rats treated with varying doses of mesembrenol or mesembranol.

FIG. 11 shows changes in seminal vesicle pressure AUC for rats treated with varying doses of mesembrenol or mesembranol.

### DETAILED DESCRIPTION

While the following terms are believed to be well understood by one of ordinary skill in the art, the following definitions are set forth to facilitate explanation of the presently disclosed subject matter.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than in another embodiment, to B only (optionally including elements other than A); in yet another embodiment, to both A and B (optionally including other elements); etc.

### Premature Ejaculation

In some embodiments, the present disclosure provides methods of treating a sexual dysfunction in a human male subject in need thereof comprising administering an effective amount of a mesembrine alkaloid or salt thereof to the subject in need thereof. In some embodiments, the sexual dysfunction is premature ejaculation.

Premature (early) ejaculation is defined in DSM-5 as a persistent or recurrent pattern of ejaculation occurring during partnered sexual activity within about one minute following vaginal penetration and/or before the individual or partner wishes it. In some embodiments, premature ejaculation occurs during sexual activities not involving vaginal penetration. In some embodiments, the subject experiences premature ejaculation on all or almost all occasions of sexual activity, The disorder may be specified by severity and can be categorized as lifelong, acquired, generalized, and situational. In some embodiments, premature (early) ejaculation is defined as ejaculation occurring about two minutes following vaginal penetration and/or before the individual or partner wishes it. In some embodiments, premature ejaculation is determined by measuring ejaculation latency. Ejaculation latency refers to the period of time between commencement of sexual activity and ejaculation.

Proper treatment of premature ejaculation involves inhibiting early ejaculation and ensuring that the patient has increased control over the timing of the ejaculation. Treatments for premature ejaculation include psychotherapeutic/behavioral therapies and pharmacological treatments (reviewed in Sharlip, J. Sex Med., Supp. 2: 103-109 (2005)). Behavioral therapy, such as the Semans pause maneuver, the Masters and Johnson pause-squeeze technique or the Kaplan stop-start method, is considered the gold standard for the treatment of premature ejaculation (Seftel, A. D., Altohob, S. E., “Premature Ejaculation”, Diagnosis and Management of Male Sexual Dysfunction, Edited by J, J. Mulcahy, New York, N.Y., Igaku-Shoin, (1997) Chapter 11, pages 196-203). While these techniques are harmless, usually painless, and are successful at rates of 60 to 95% (Seftel et al., Behay. Res. Ther., 24:377 (1986)), they require partner cooperation and improvement is short-lived (Bancroft, J. and Coles, L., Brit. Med. J., 1:1575 (1976) and De Amicus, L. A., et al., Arch. Sex. Behay., 14:467 (1985)).

Pharmacological agents reported to provide some desirable delay in ejaculation in the male patient include topical anesthetics, non-selective serotonin reuptake inhibitors (SRIs), and selective serotonin reuptake inhibitors (SSRIs). Topical anesthetics, e.g., preparations containing benzocaine or a lidocaine-prilocaine combination, are applied to the penis to diminish sensitivity and delay ejaculation. However, topically applied anesthetics can cause irritation to either or both partners or result in an undesired hypoesthesia that prevents orgasm.

SSRIs have also been reported to delay or inhibit sexual climax and to delay ejaculation. Such sexual side effects, including delay of ejaculation, have been reported for such well known antidepressants as fluoxetine (commercially available as PROZAC®, Eli Lilly and Company, Indianapolis, Ind.), sertraline (commercially available as PAXIL®, GlaxoSmithKline plc, London, United Kingdom), paroxentine (commercially available as ZOLOFT®, Pfizer Inc., New York, N.Y.), and dapoxetine (Alza Corporation, Mountain View, Calif). Accordingly, SSRIs have also been the focus for developing treatments for premature ejaculation. Similar sexual side effects have been noted in patients treated with the tricyclic antidepressant clomipramine (e.g., ANAFRANIL®, Novartis International AG, Basel, Switzerland), which has also been used to treat premature ejaculation PE. However, SSRIs have common several side effects, including feeling agitated, shaky or anxious, indigestion, diarrhea or constipation, loss of appetite and weight loss, dizziness, blurred vision, dry mouth, excessive sweating, sleeping problems (insomnia) or drowsiness, headaches, low sex drive, difficulty obtaining or maintaining an erection.

Additional sexual dysfunctions include low sex drive or decreased libido. In some embodiments, the present disclosure provides methods of treating a sexual dysfunction in a human male subject in need thereof comprising administering an effective amount of a mesembrine alkaloid or salt thereof to the subject in need thereof, wherein the sexual dysfunction is decreased libido.

### Orgasm

In some embodiments, the present disclosure provides methods of delaying orgasm in a human male subject comprising administering an effective amount of a mesembrine alkaloid or salt thereof to the subject. In some embodiments, the human male subject suffers from a sexual dysfunction (e.g., premature ejaculation). In some embodiments, the human male subject does not suffer from a sexual dysfunction. As used herein, “orgasm” or “climax” (used interchangeably herein) refer to the sudden discharge of accumulated sexual excitement during the sexual response cycle, resulting in rhythmic muscular contractions in the pelvic region characterized by sexual pleasure. Human orgasms usually result from physical sexual stimulation of the penis in males (typically accompanying ejaculation) and of the clitoris in females. Sexual stimulation can be by self-practice (masturbation) or with a sex partner (penetrative sex, non-penetrative sex, or other sexual activity). Orgasms are experienced by males and females and are controlled by the involuntary or autonomic nervous system. They are usually associated with involuntary actions, including muscular spasms in multiple areas of the body, a general euphoric sensation and, frequently, body movements and vocalizations. ejaculation.

The term “climax control” refers to the delay of orgasm and/or the delay of

### Orgasm in Males

In men, the most common way of achieving orgasm is by physical sexual stimulation of the penis, although orgasm can also be achieved by stimulation of the prostate. Orgasm in men is usually accompanied by ejaculation, but it is possible for men to orgasm without ejaculation (known as a “dry orgasm”). For example, prepubescent boys have dry orgasms. Dry orgasms can also occur as a result of retrograde ejaculation, or hypogonadism. Men may also ejaculate without reaching orgasm, which is known as anorgasmic ejaculation.

### Mesembrine Compounds

In some embodiments, the present disclosure provides methods of treating a sexual dysfunction in a human male subject in need thereof comprising administering an effective amount of a mesembrine alkaloid or salt thereof to the subject in need thereof. In some embodiments, the sexual dysfunction is premature ejaculation.

Mesembrine is an alkaloid present in Scelethium tortuosum—a succulent plant commonly found in South Africa and also known as Kanna, Channa, Kougoed. S. tortuosum contains about 1-1.5% total alkaloids. Mesembrine alkaloids, including mesembrine, mesembrenone, mesembrenol and tortuosamine, can he isolated from plants found in the family Aizoaceae. Mesembrine compositions are described in WO 1997/046234, which is incorporated herein by reference. Nilesembrine compounds have been shown to inhibit phosphodiesterase-4 (PDE4) subtypes and serotonin transporters. Without wishing to be bound by theory, this dual inhibition activity may be responsible for the unexpected delay in ejaculation.

In some embodiments, the mesembrine alkaloid or salt thereof is an extract from the Sceletium tortnosum plant. In some embodiments, the mesembrine alkaloid extract is a substantially pure extract. The term “substantially pure” means preparations of mesembrine alkaloids having a chromatographic purity of greater than about 95%. In some embodiments, the chromatographic purity is greater than 96%. In some embodiments, the chromatographic purity is greater than about 97%. In some embodiments, the chromatographic purity is greater than about 98%. In some embodiments, the chromatographic purity is greater than about 99%. In some embodiments, the chromatographic purity is greater than about 99.5%.

In some embodiments, the mesembrine alkaloid is chemically synthesized using the following methods.

As shown in Scheme 1 below, and described in Pinnick et al., Tetrahedron Letters, 1983, 24, 4785-4788, and Pinnick et al., J. Org. Chem. 1987, 52, 2111 which are incorporated herein by reference, mesembrine can be prepared by reacting 1-methylpyrrolidin-2-one (1-1) with an amide base such as lithium diisopropylamide (LDA), lithioisopropylcyclohexylamine (LICA), or lithium 2,2,6,6-tetramethylpiperidide (LTMP) in a cooled solution of an ether solvent such as THF (e.g., in the range of 0° C. to −78° C.), followed by the addition of 4-bromoveratrole (1-2) to give lactam 1-3. Lactam 1-3 is reduced with DIBAL-H to form enamine 1-4 which is reacted with methyl vinyl ketone to form mesembrine, which is purified by chromatography on silica gel.

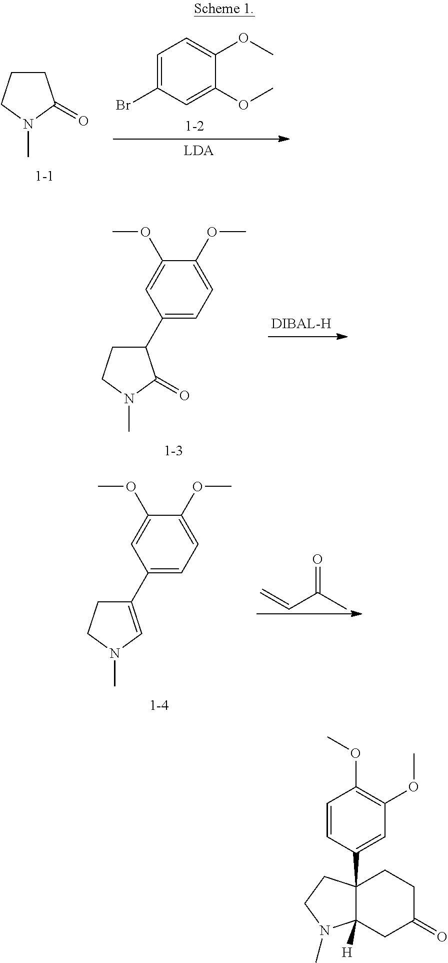

Mesembrine can also be prepared for example, via the enantioselective synthetic route shown Scheme 2 below for the synthesis of (−)-mesembrine, and described in Taber et al., J. Org. Chem 2005, 70, 7711-7714, which is incorporated herein by reference.

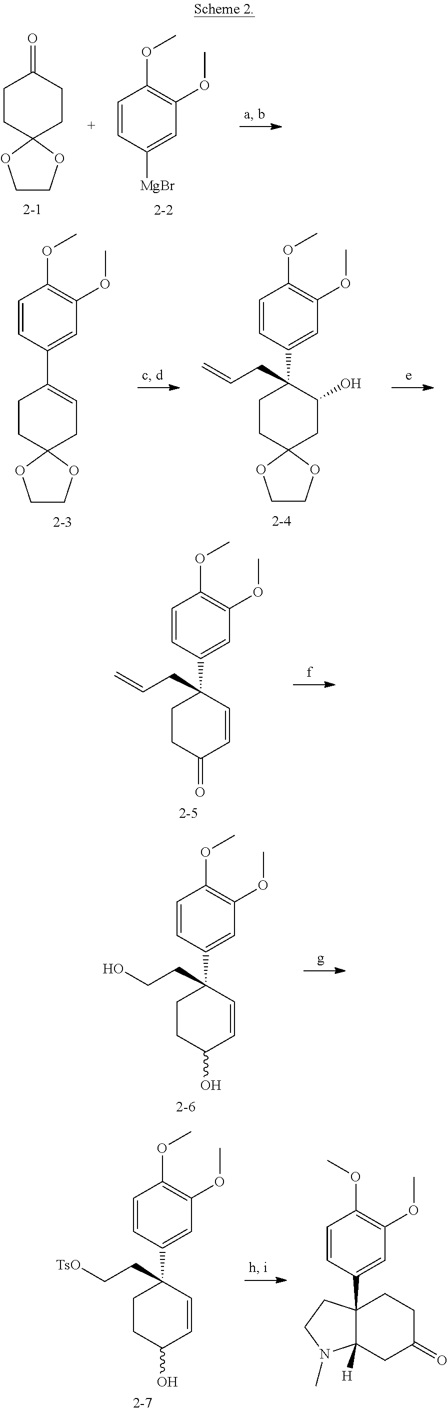

(a) THF, 0-20° C., overnight; (b) benzene, PTSA (cat.), ethylene glycol (excess), reflux; (c) Shi's catalyst, DME-acetonitrile-H₂O, 0° C., 4 h; (d) al lylmagnesium chloride, THE, 0-20° C., overnight; (e) 10% aqueous HCl, THF, reflux, 1 h; (f) O₃, MeOH, −78° C.; CeCl₃.7H₂O (1.0 equiv), NaBH₄ (8.0 equiv), 0° C.; (g) TsCl (1 equiv), Et₃N, CH₂Cl₂, 20° C., overnight; (h) 40% aqueous CH₃NH₂, THF, 65° C., 1 h; (i) MnO₂, CH₂Cl₂, 20° C., 3 h.

In embodiments, the mesembrine alkaloid is a salt, including those obtained by reacting the mesembrine alkaloid functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid. benzoic acid, tartaric acid, fumaric acid, salicylic acid, mandelic acid, carbonic acid, etc. Those skilled in the art will further recognize that acid addition salts may be prepared by reaction of the mesembrine alkaloid with the appropriate inorganic or organic acid via any of a number of known methods. In some embodiments, the mesembrine alkaloid is mesembrine HCl.

Mesembrine alkaloids include mesembrine, mesembrenone, Δ⁷mesembrenone, 4′-O-demethymesembranol, mesembrenol, 4′-O-demethymesembrenol, mesembranol, 4′-O-demethylmesembrenone, sceletenone, N-demethly-N-formulmesembrenone, O-acetylmesembrenol, mesembrane; N-demethelymesembrenol; N-demethylmesembranol, hordenine, joubertiamine, dehydrojoubertiamine, dehydrojoubertiamine, joubertinamine, O-methydehydrojoubertiamine, O-methyljoubertiamine, O-methyldihydrojoubertiamine, 3′-methoxy-4′-O-methyoubertiamine, 3-methoxy-4′O-methyljoubertiaminol, dimethoxyphenyl-4-[2-acetylmethylamino)ethyl]cyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine.

In some embodiments, the mesembrine alkaloid is selected from mesembrine, mesembrenone, Δ⁷mesembrenone, 4′-O-demethylmesembranol, mesembrenol, 4′-O-demethylmesembrenol, mesembranol, 4′-O-demethylmesembrenone, sceletenone, N-demethly-N-formulmesembrenone, O-acetylmesembrenol, mesembrane, N-demethelymesembrenol, N-demethylmesembranol, hordenine, joubertiamine, dehydrojoubertiamine, dehydrojoubertiamine, joubertinamine, O-methyldehydrojoubertiamine, O-methyljoubertiamine, O-methyldihydrojoubertiamine, 3-methoxy-4′-O-methyljoubertiamine, 3′-methoxy-4′O-methyljoubertiaminol, 4-(3,4-dimethoxyphenyl-4-acetylmethylamino)ethylicyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine. In some embodiments, the mesembrine alkaloid is selected from mesembrine, mesembrenone, mesembrenol, and tortuosamine.

Non-limiting embodiments of meserabrine alkaloids include:

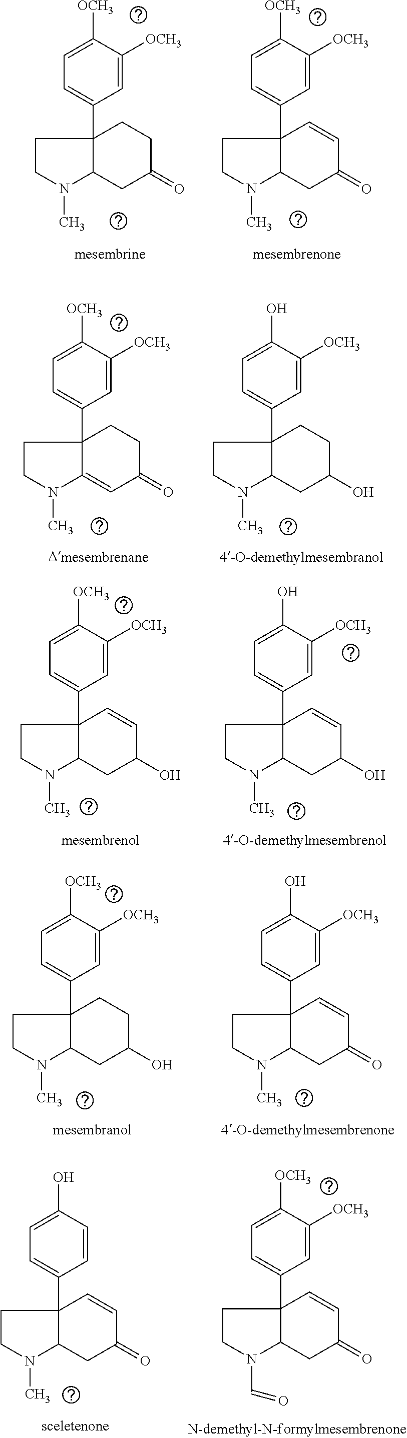

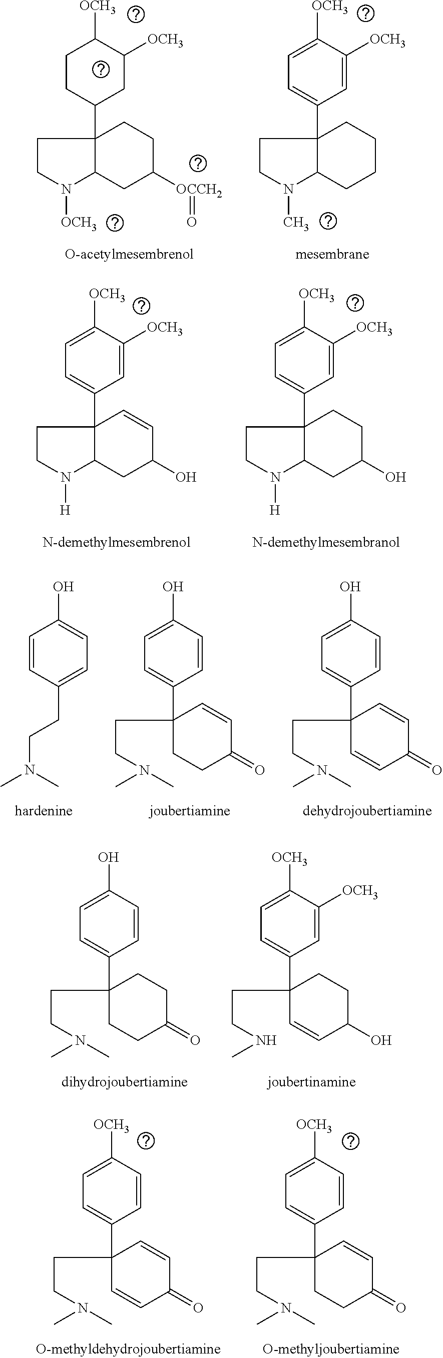

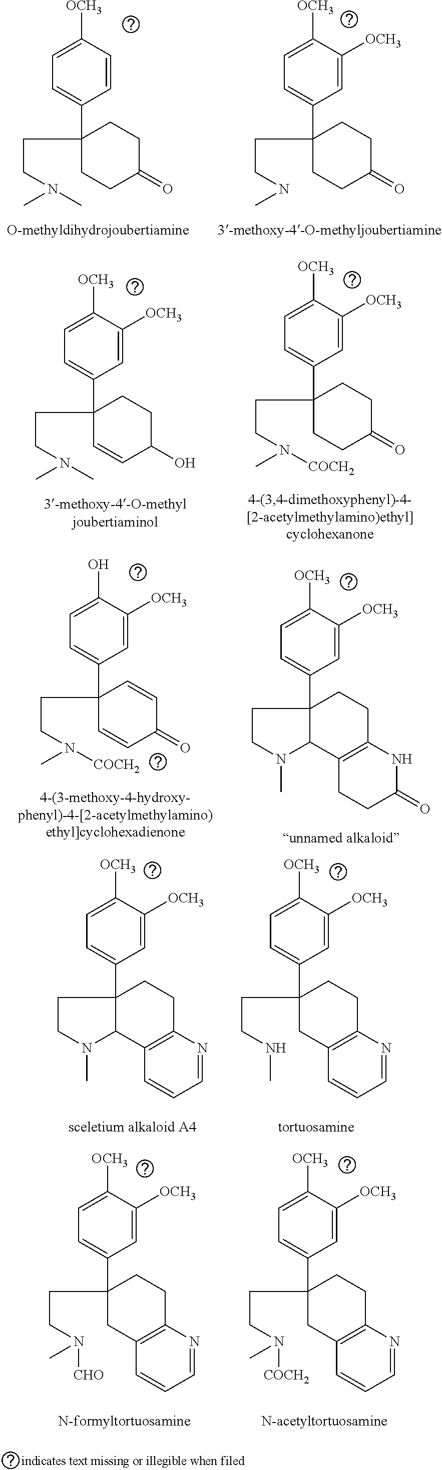

In some embodiments, the mesembrine alkaloid is a compound of Formula (I):

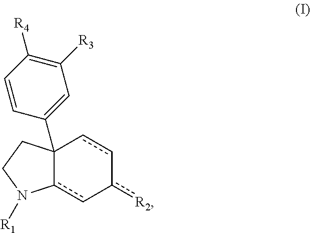

or a salt thereof
wherein,
Rₜ is H, CH₃, CHO, OCH₃;
R₂ is H, OH, O-acetyl, or O; and
R₃ and R₄ are each independently H, OH, or methoxy

In some embodiments of the compound of Formula (I), R³ is methoxy and R⁴ is OH.

In sonic embodiments of the compound of Formula (I), R³ and R⁴ are methoxy.

In sonic embodiments of the compound of Formula (I), R₁ is H or CH₃. In sonic embodiments, R₁ is CH₃. In some embodiments, R₁ is H.

In some embodiments of the compound of Formula (I), R₂ is H, OH, or O. In some embodiments, R₂ is H. In embodiments, R₂ is OH. In some embodiments, R₂ is O.

In some embodiments, the mesembrine compounds described herein are racemic mixtures. In some embodiments, the mesembrine compounds described herein are non-racemic mixtures (i.e., mixtures where one enantiomer is more abundant than the other). As understood in the art, the composition of such mixtures can be described by its enantiomeric excess. In some embodiments, the mesembrine compounds described herein have an enantiomeric excess % ee) of about 99%, about 98%, about 97%, about 96%, about 95%, about 90%, about 85%, about 80%, about 75%, about 70%, about 65%, about 60%, about 55%, about 50%, about 45%, about 40?, about 35%, about 30%, about 25%, about 20%, about 15%, about 10%, or about 5% ee, including all ranges and values therebetween. In some embodiments, the mesembrine compounds described herein are enantiopure, In some embodiments, an enantiopure mesembrine compound has an enantiomeric excess (% ee) greater than about 50%, greater than about 55%, greater than about 60%, greater than about 65%, greater than about 70%, greater than about 75%, greater than about 80%, greater than about 85%, greater than about 90%, greater than about 95%, or greater than about 99%, including all ranges and values therebetween.

In some embodiments, a mesembrine compound described herein having two or more chiral centers is a mixture of diastereomers. In some embodiments, a mesembrine compound having two or more chiral centers is diastereomerically pure. In some embodiments, a diastereomerically pure mesembrine compound has a diastereomeric excess (de) of greater than about 50%, greater than about 55%, greater than about 60%, greater than about 65%, greater than about 70%, greater than about 75%, greater than about 80%, greater than about 85%, greater than about 90%, greater than about 95%, or greater than about 99%.

For administration, the mesembrine compounds described herein may be formulated as a pharmaceutical composition. A pharmaceutical composition may comprise: (i) a mesembtine compound; and (ii) a pharmaceutically acceptable carrier, diluent, or excipient. A pharmaceutical composition comprising a mesembrine compound described herein can be formulated according to known methods to prepare pharmaceutically useful compositions, whereby the therapeutic molecule is combined in a mixture with a pharmaceutically acceptable carrier, diluent, or excipient.

A carrier is said to be a “pharmaceutically acceptable carrier” if its administration can be tolerated by a recipient patient. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising a mesembrine compound described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, a liquid, a lozenge, a fast disintegrating tablet, a lyophilized preparation, a film, a spray (including a nasal spray, an oral spray, or a topical spray), or a mucoadhesive. The oral unit dosage form may be selected from the group consisting of: tablets, pills, pellets, capsules, powders, lozenges, granules, solutions, suspensions, emulsions, syrups, elixirs, sustained-release formulations, aerosols, and sprays. In some embodiments, the mesembrine compound is formulated as a liquid, a lozenge, a fast disintegrating tablet, a lyophilized preparation, a film, a spray, or a mucoadhesive.

Pharmaceutical compositions comprising mesembrine alkaloids as described herein may also contain one or more additional ingredients including, but not limited to, a mucoadhesive compound, a buffering agent, a plasticizing agent, a stabilizing agent, a taste-masking agent, a flavoring agent, a coloring agent, an antiseptic, an inert filler agent, a preservative, and combinations thereof.

In some embodiments, the formulations comprise one or more solubilizing agents that increase the solubility of mesembrine in the formulation. Suitable solubilizing agents are known to those skilled in the art and include, for example, complexing agents, surfactants, and the like. Suitable complexing agents include unsubstituted cyclodextrins (such as alpha-cyclodextrin, beta-cyclodextrin) and substituted cyclodextrins, (such as hydroxypropyl beta-cyclodextrin, sulfobutylether-beta-cyclodextrin). Suitable surfactants include polyoxyethylene sorbitan monolaurate (for example, Tween 20), polyoxyethylene sorbitans molooleate (for example, Tween 80), polyethylene glycol (15)-hydroxystearate (for example, Kolliphor® HS 15), PEG-35 castor oil (for example, Kolliphor® EL) and PEG-60 hydrogenated castor oil (for example, Cremophorg RH 60).

In some embodiments, the formulations comprise one or more buffer agents that maintain the pH of the IV solution within a pharmaceutically acceptable range. In certain embodiments, the buffer maintains the pH of the IV solution between about 5 and 9. In specific embodiments, the buffer maintains the pH of the IV solution at about 7.4. Suitable buffers are known to those skilled in the art and include, for example, citrates, lactate, acetate, maleate, phosphates, and the like.

In some embodiments, the formulations comprise one or more density modifiers that is used to control the density of the IV formulation. Suitable density modifiers are known to those skilled in the art and include, for example, dextrose.

In some embodiments, the formulations comprise one or more isotonicity modifiers that provide a formulation that is iso-osmotic with tissue to prevent pain and irritation when the formulation is administered. Suitable isotonicity modifiers are known to those skilled in the art and include, for example, electrolytes, monosaccharides, and disaccharides. Examples of isotonicity modifiers include glycerin, dextrose, potassium chloride: and sodium chloride.

In some embodiments, the formulations comprise one or more viscosity enhancers. Suitable viscosity enhancers are known to those skilled in the art and include, for example, povidone, hydroxyethylcellulose, hydroxymethylcellulose, polyvinyl alcohol and carbomer (such as, acrylic acid homopolymers and acrylic acid copolymers).

In some embodiments, the formulations comprise one or more preservatives that increase the stability of mesembrine in the formulation and/or provide antimicrobial activity. Suitable preservatives are known to those skilled in the art and include, for example, antimicrobial agents and antioxidants. Examples of antimicrobial agents (with ranges of anti-microbial effective amounts shown as weight of antimicrobial agent per volume of IV formulation, i.e., % w/v) include benzyl alcohol (about 0.1-3.0% w/v”), methyl paraben (about 0.08-0.1% w/v), propyl paraben (0.001-0.023% w/v), phenol (0.2-0.5% w/v), cresol (0.2-0.5% w/v) methyl paraben (0.1% w/v), chlorbutanol (0.25-0.5% w/v), sodium metabisulphite (0.025-0.66% w/v), sodium bisulphite (0.13-0.2% w/v), benzethonium chloride (0.08-0.1% w/v), and benzalkonium chloride (0.08-0.1% w/v). Examples of antioxidants include sodium bisulphite and other sulfurous acid salts, ascorbic acid, salts of ethylenediaminetetraacetic acid (including sodium), alpha tocoherol butylated hydroxyl hydroxytoluene, and butylated hydroxyanisole.

A pharmaceutical composition comprising a mesembrine compound described herein may be administered to a subject in a therapeutically effective amount. According to the methods of the present disclosure, a mesembrine compound described herein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, a mesembrine compound described herein can be administered to a subject in a single bolus delivery, via continuous delivery (e.g., continuous transdermal delivery) over an extended time period, or in a repeated administration protocol (e.g., on an hourly, daily, weekly, or monthly basis).

Pharmaceutical compositions comprising the mesembrine compound described herein can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid, aerosol generator, or nebulizer for administration of a pharmaceutical composition. Such a kit can further comprise written information on indications and usage of the pharmaceutical composition.

### Methods of Treatment

In some embodiments, the present disclosure provides methods of treating premature ejaculation in a human male comprising administering a mesembrine alkaloid or salt thereof to the male in need thereof. In some embodiments, the present disclosure provides methods of delaying orgasm in a human male comprising administering a mesembrine alkaloid or salt thereof to the male in need thereof. In some embodiments, the human male suffers from a sexual dysfunction. In some embodiments, the human male is a healthy male.

In some embodiments, the methods provided herein comprise administration of the mesembrine alkaloid or salt thereof to the male in need thereof prior to commencement of sexual activity. The term “sexual activity” refers to any activity solitary, between two persons, or in a group—that induces sexual arousal. Examples of sexual activity include, but are not limited to, masturbation, mutual masturbation, sexual intercourse, vaginal sex, anal sex, oral sex, fellatio non-penetrative sex, genital rubbing, manual stimulation, and digital stimulation. In some cases, the sexual activity may include the use of devices, toys, or other inanimate aids.

In some embodiments, the present disclosure provides methods for enhancing one or more aspects of sexual activity in a human male comprising administering a mesembrine alkaloid or salt thereof to the male in need thereof. In some embodiments, the human male suffers from a sexual dysfunction. In some embodiments, the human male is a healthy male. In some embodiments, the one or more aspects may be increased or enhanced control over climax (including, but not limited to, delay of ejaculation, delay of climax, control of climax, prolonging of climax, and achieve mutual climax). In sonic embodiments, the one or more aspects may be increased or enhanced control over orgasm (including, but not limited to, delay of orgasm, control of orgasm, prolonging of orgasm, and achieve mutual orgasm). In some embodiments, the one or more aspects may be an increase in or enhancement of perceived pleasure of the subject and/or the subject's partner (including but not limited to, extended or prolonged feelings of pleasure during sexual activity or increased or enhanced sexual satisfaction). In some embodiments, the one or more aspects may be enhanced or improved sexual performance (including, but not limited to, increased libido, increased sexual satisfaction of partner, or overall extension of time to climax during sexual activity). In some embodiments, the subject may wish to delay ejaculation. In some embodiments, the subject may wish to delay orgasm. In some cases, ejaculation is achieved without orgasm. In some cases, orgasm is achieved without ejaculation. in some cases, ejaculation and orgasm occur simultaneously.

In some embodiments, the methods provided herein do not result in a concurrent increase or onset of negative symptoms such as low sex drive or difficulty obtaining or maintaining an erection. Additional negative symptoms may include feeling agitated, shaky or anxious, indigestion, diarrhea or constipation, loss of appetite and weight loss, dizziness, blurred vision, dry mouth, excessive sweating, sleeping problems (insomnia) or drowsiness, and/or headaches.

Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Accordingly, a “therapeutically effective amount,” as used herein, refers to an amount of a compound is an amount that achieves the desired biologic or therapeutic effect, namely an amount that prevents, reduces, or ameliorates one or more symptoms of the enumerated diseases being treated or prevented. For example, the therapeutically effective amount of the mesembrine compound will depend on the condition to be treated, the severity and course of the condition, whether the mesembrine compound is administered for preventive or therapeutic purposes, previous therapy, the patient's clinical history and response to the mesembrine compound, and the discretion of the attending physician. The mesembrine compound described herein is suitably administered to the patient at one time or over a series of treatments and may be administered to the patent at any time from diagnosis onwards. The mesembrine compound described herein may be administered as the sole treatment or in conjunction with other drugs or therapies useful in treating the condition in question.

In some embodiments, the therapeutically effective amount of the mesembrine alkaloid or salt thereof is at least 0.001 mg. In some embodiments, the therapeutically effective amount of the mesembrine alkaloid or salt thereof is determined from pre-clinical animal studies. Guidance for the conversion of animal doses to human equivalent doses is provided in the FDA Guidance For Industry Document entitled “Estimating the Maximum Safe Starting Dose in Initial Clinical Trials for Therapeutics in Adult Healthy Volunteers” released July 2005, which is incorporated herein by reference, Table I from this document, which provides the conversion factors for converting between human and animal dosing, is excerpted below.

TABLE 1
Conversion of Animal Doses to Human Equivalent Doses
Based on Body Surface Area

| | To Convert | | |
|---|---|---|---|
| | Animal Dose in | To Convert Animal Dose in mg/kg | |
| | mg/kg to Dose in | to HED^{a} in mg/kg, Either: | |
| | mg/m², Multiply | Divide | Multiply |
| Species | by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, thesus, and stumptail.

In some embodiments, the therapeutically effective amount of the mesembrine alkaloid or salt thereof is between 0.001 and 0.010 mg. In some embodiments, the therapeutically effective amount of the mesembrine alkaloid or salt thereof is at least 0.0015, 0.002 mg, 0.0025 mg, 0.003 mg, 0.0035 mg, 0.004 mg, 0.0045 mg, 0.005 mg, 0.0055 mg, 0.006 mg, 0.0065 mg, 0.007 mg, 0.0075 mg, 0.008 mg, 0.0085 mg, 0.009 mg, 0.0095 mg, 0.010 mg. In some embodiments, the therapeutically effective amount of the mesembrine alkaloid or salt thereof is at least 0.01 mg. In some embodiments, the therapeutically effective amount of the mesembrine alkaloid or salt thereof is between about 0.01 mg and 1 mg. In some embodiments, the therapeutically effective amount of the mesembrine alkaloid or salt thereof is at least 0.01 mg, 0.015 mg, 0.02 mg, 0.025 mg, 0.03 mg, 0.035 mg, 0.4 mg, 0.045 mg, 0.05 mg, 0.055 mg, 0.06 mg, 0.065 mg, 0.07 mg, 0.075 mg, 0,08 mg. 0.085 mg, 0.09 mg, 0.095 mg, or at least 0.01 mg. In some embodiments, the therapeutically effective amount of the mesembrine alkaloid or salt thereof is at least 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, or at least 1 mg. In some embodiments, the therapeutically effective amount of the mesembrine alkaloid or salt thereof is between 0.01 mg and 500 mg. In some embodiments, the therapeutically effective amount of the mesembrine alkaloid or salt thereof is between 500 mg and 1000 mg. In some embodiments, the therapeutically effective amount of the mesembrine alkaloid or salt thereof is between 0.01 mg and 1 mg.

Disclosures of such ranges herein intended to be a disclosure of all intervals within this range. For example, a disclosure of between 0.01 mg and 1 mg is a disclosure of 0.01, 0.02, 0.03, 0.04, 0.05, 0.06, 0.07, 0.08, 0.09, 0.11, 0.12, 0.13, 0.14, 0.15, 0.16, 0.17, 0.18, 0.19, 0.2. 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0,58, 0.59, 0.6, 0.61, 0.62. 0.63. 0.64. 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87 0.88 0.89, 0.9, 0.91, 0.92, 0.93, 0.94, 0.95, 0.96, 0.97, 0.98, 0.99, and 1 mg.

In some embodiments, the therapeutically effective amount of the mesembrine alkaloid or salt thereof is between 1 mg and 10 mg. In some embodiments, the therapeutically effective amount of the mesembrine alkaloid or salt thereof is between 10 mg and 20 mg. In some embodiments, the therapeutically effective amount of the mesembrine alkaloid or salt thereof is between 20 mg and 30 mg. In some embodiments, the therapeutically effective amount of the mesembrine alkaloid or salt thereof is between 30 mg and 40 mg. In some embodiments, the therapeutically effective amount of the mesembrine alkaloid or salt thereof is between 40 mg and 50 mg.

In some embodiments, the therapeutically effective amount of the mesembrine alkaloid or salt thereof is between 50 mg and 100 mg. In some embodiments, the therapeutically effective amount of the mesembrine alkaloid or salt thereof is between 100 mg and 150 mg. In some embodiments, the therapeutically effective amount of the mesembrine alkaloid or salt thereof is between 150 mg and 200 mg. In some embodiments, the therapeutically effective amount of the mesembrine alkaloid or salt thereof is between 200 mg and 250 mg. In some embodiments, the therapeutically effective amount of the mesembrine alkaloid or salt thereof is between 250 mg and 300 mg. In some embodiments, the therapeutically effective amount of the mesembrine alkaloid or salt thereof is between 300 mg and 350 mg. In some embodiments, the therapeutically effective amount of the mesembrine alkaloid or salt thereof is between 350 mg and 400 mg. In some embodiments, the therapeutically effective amount of the mesembrine alkaloid or salt thereof is between 450 mg and 500 mg.

In some embodiments, the therapeutically effective amount of the mesembrine alkaloid or salt thereof is between 500 mg and 550 mg. In some embodiments, the therapeutically effective amount of the mesembrine alkaloid or salt thereof is between 550 mg and 600 mg. In some embodiments, the therapeutically effective amount of the mesembrine alkaloid or salt thereof is between 600 mg and 650 mg. In some embodiments, the therapeutically effective amount of the mesembrine alkaloid or salt thereof is between 650 mg and 700 mg. In some embodiments, the therapeutically effective amount of the mesembrine alkaloid or salt thereof is between 700 mg and 750 mg, In some embodiments, the therapeutically effective amount of the mesembrine alkaloid or salt thereof is between 750 mg and 800 mg. In some embodiments, the therapeutically effective amount of the mesembrine alkaloid or salt thereof is between 800 mg and 850 mg, In some embodiments, the therapeutically effective amount of the mesembrine alkaloid or salt thereof is between 850 mg and 900 mg. In some embodiments, the therapeutically effective amount of the mesembrine alkaloid or salt thereof is between 900 mg and 950 mg. In some embodiments, the therapeutically effective amount of the mesembrine alkaloid or salt thereof is between 950 mg and 1000 mg.

In some embodiments, the therapeutically effective amount of the mesembrine alkaloid or salt thereof is between 1 μg/kg and 10 μg/kg, between 10 μg/kg and 20 μg/kg, between 20 μg/kg and 30 μg/kg, between 30 μg/kg and 40 μg/kg, between 40 μg/kg and 50 μg/kg, between 50 μg/kg and 60 μg/kg, between 60 μg/kg and 70 μg/kg, between 70 μg/kg and 80 μg/kg, between 80 μg/kg and 90 μg/kg, between 90 μg/kg and 100 μg/kg. In some embodiments, the therapeutically effective amount of the mesembrine alkaloid or salt thereof is 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 31 μg/kg, 32 μg/kg, 33 μg/kg, 34 μg/kg, 35 μg/kg, 36 μg/kg, 37 μg/kg, 38 μg/kg, 39 μg/kg, 40 μg/kg, 41 μg/kg, 42 μg/kg, 43 μg/kg, 44 μg/kg, 45 μg/kg, 46 μg/kg, 47 μg/kg, 48 μg/kg, 49 μg/kg, 50 μg/kg, 51 μg/kg, 52 μg/kg, 53 μg/kg, 54 μg/kg, 55 μg/kg, 56 μg/kg, 57 rig/kg 58 μg/kg, 59 μg/kg, 60 μg/kg, 61 μg/kg, 62 μg/kg, 63 μg/kg, 64 μg/kg, 65 μg/kg, 66 μg/kg, 67 μg/kg, 68 μg/kg, 69 μg/kg 70 μg/kg, 71 μg/kg, 72 μg/kg, 73 μg/kg, 74 μg/kg, 75 μg/kg, 76 μg/kg, 77 μg/kg, 78 μg/kg, 79 μg/kg, 80 μg/kg, 81 μg/kg 82 μg/kg 83 μg/kg 84 μg/kg 85 μg/kg, 86 g/kg 87 μg/kg 88 μg/kg 89 μg/kg, 90 μg/kg, 91 μg/kg, 92 μg/kg, 93 μg/kg, 94 μg/kg, 95 μg/kg, 96 μg/kg, 97 μg/kg, 98 μg/kg, 99 μg/kg, or 100 μg/kg. In some embodiments, the therapeutically effective amount of the mesembrine alkaloid or salt thereof is 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, or 1000 μg/kg.

In some embodiments, the therapeutically effective amount of the mesembrine alkaloid or salt thereof is about 3 μg/kg. In some embodiments, the therapeutically effective amount of the mesembrine alkaloid or salt thereof is about 2.5 μg/kg, 2.6 μg/kg, 2.7 μg/kg, 2.8 μg/kg, 2.9 μg/kg, 3.0 μg/kg, 3.1 μg/kg, 3.2 μg/kg, 3.3 μg/kg, 3.4 μg/kg, 3.5 μg/kg, 3.6 μg/kg, 3.7 μg/kg, 3.8 μg/kg, 3.9 μg/kg, or 4 μg/kg. In some embodiments, the therapeutically effective amount of the mesembrine alkaloid or salt thereof is about 3.2 μg/kg. In some embodiments, the therapeutically effective amount of the mesembrine alkaloid or salt thereof is about 30 μg/kg. In some embodiments, the therapeutically effective amount of the mesembrine alkaloid or salt thereof is about 25 μg/kg, 26 μg/kg, 27 μg/kg, 28 μg/kg, 29 μg/kg, 30 μg/kg, 31 μg/kg, 32. μg/kg μg/kg, 34 μg/kg, 35 μg/kg, 36 μg/kg. In some embodiments, the therapeutically effective amount of the mesembrine alkaloid or salt thereof is about 32 μg/kg. In some embodiments, the therapeutically effective amount of the mesembrine alkaloid or salt thereof is about 300 μg/kg. In some embodiments, the therapeutically effective amount of the mesembrine alkaloid or salt thereof is about 250 μg/kg, 260 μg/kg, 270 μg/kg, 280 μg/kg, 290 μg/kg, 300 μg/kg, 310 μg/kg, 320 μg/kg 330 μg/kg, 340 μg/kg, or 350 μg/kg. In some embodiments, the therapeutically effective amount of the mesembrine alkaloid or salt thereof is about 320 μg/kg.

In some embodiments, the therapeutically effective amount of the mesembrine alkaloid or salt thereof is about 1.5 mg/kg. In some embodiments, the therapeutically effective amount of the mesembrine alkaloid or salt thereof is about 1.0 mg/kg, 1.1 mg/kg, 1.2. mg/kg, 1.3 mg/kg, 1.4 mg/kg, 1.5 mg/kg, 1.6 mg/kg, 1.7 mg/kg, 1.8 mg/kg, 1.9 mg/kg, or 2.0 mg/kg. In some embodiments, the therapeutically effective amount of the mesembrine alkaloid or salt thereof is about 1.6 mg/kg. In some embodiments, the therapeutically effective amount of the mesembrine alkaloid or salt thereof is about 3 mg/kg. In some embodiments, the therapeutically effective amount of the mesembrine alkaloid or salt thereof is about 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, or 3.5 mg/kg. In sonic embodiments, the therapeutically effective amount of the mesembrine alkaloid or salt thereof is about 3.2 mg/kg.

In some embodiments, the therapeutically effective amount of the mesembrine alkaloid or salt thereof is between 1 mg/kg and 10 mg/kg, between 10 mg/kg and 20 mg/kg, between 20 mg/kg and 30 mg/kg, between 30 mg/kg and 40 mg/kg, between 40 mg/kg and 50 mg/kg, between 50 mg/kg and 60 mg/kg, between 60 mg/kg and 70 mg/kg, between 70 mg/kg and 80 mg/kg, between 80 mg/kg and 90 mg/kg, between 90 mg/kg and 100 mg/kg. In some embodiments, the therapeutically effective amount of the mesembrine alkaloid or salt thereof is 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg 61 mg/kg, 62 mg/kg, 63 mg/kg, 64 mg/kg 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, or 100 mg/kg.

In some embodiments, the therapeutically effective amount of the mesembrine alkaloid or salt thereof is 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg.

In some embodiments, treating premature ejaculation comprises reducing the frequency at which premature ejaculation occurs. In some embodiments, treating premature ejaculation comprises increasing the amount of time after commencement of sexual activity and before ejaculation. In some embodiments, treating premature ejaculation comprises increasing the amount of time after commencement of sexual activity and before ejaculation to greater than 1 minute. In some embodiments, treating premature ejaculation comprises increasing the amount of time after commencement of sexual activity and before ejaculation to greater than 2, 3, 4, 5, 6, 7, 8, 9, 10, 11, 12, 13, 14, 15, 16, 17, 18, 19, 20, 21, 22, 23, 24, 25, 26, 27, 28, 29, 30 minutes or more.

In some embodiments, delaying orgasm comprises increasing the amount of time after commencement of sexual activity and before ejaculation and/or orgasm. In some embodiments, delaying orgasm comprises increasing the amount of time after commencement of sexual activity and before ejaculation and/or orgasm to greater than 1 minute. In sonic embodiments, delaying orgasm comprises increasing the amount of time after commencement of sexual activity and before ejaculation and/or orgasm to greater than 2, 3, 4, 5, 6, 7, 8, 9, 10, 11, 12, 13, 14, 15, 16, 17, 18, 19, 20, 21, 22, 23, 24, 25, 26, 27, 28, 29, 30 minutes or more.

In some embodiments, treating premature ejaculation and/or delaying orgasm comprises increasing the intravaginal ejaculatory latency time (IELT) to greater than one minute. In some embodiments, treating premature ejaculation and/or delaying orgasm comprises increasing the IELT to greater than 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes. 26 minutes, 27 minutes, 28 minutes, 29 minutes, 30 minutes, or more as compared to the IELT observed in the absence of treatment with a mesembrine alkaloid or salt thereof.

In some embodiments, delay in ejaculation is indicated by a decrease in seminal vesicle pressure. In some, embodiments, seminal vesicle pressure is decreased by at least 1%, 2%, 3%, 4%, 5%, 10%, 20%, 30%, 40%, 50%, 60%, 70%, 80%, 90%, 95%, 99%, or more compared to the seminal vesicle pressure observed in the absence of treatment with a mesembrine alkaloid or salt thereof.

In some embodiments, the present disclosure provides a method treating premature ejaculation and/or delaying orgasm in a human male comprising administering a mesembrine alkaloid or salt thereof to the male in need thereof, wherein the mesembrine alkaloid or salt thereof is administered prior to commencement of sexual activity. In some embodiments, the mesembrine alkaloid or salt thereof is administered at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 20 minutes, at least 30 minutes, at least 40 minutes, at least 50 minutes, or at least 60 minutes prior to commencement of sexual activity. In some embodiments, the mesembrine alkaloid or salt thereof is administered at least 1 hours, at least 2 hours, at least 3 hours, at least 4 hours, at least 5 hours, at least 6 hours, at least 7 hours, or at least 8 hours prior to commencement of sexual activity. In some embodiments, the mesembrine alkaloid or salt thereof is administered to the subject at least once daily. In some embodiments, the mesembrine alkaloid or salt thereof is administered to the subject at least twice, at least three times daily, at least four times daily, or at least 5 times daily. In some embodiments, the mesembrine alkaloid or salt thereof is administered to the subject at least once per week, In some embodiments, the mesembrine alkaloid or salt thereof is administered to the subject at least once per month.

In some embodiments, the mesembrine alkaloid is administered at commencement of sexual activity. In some embodiments, the mesembrine alkaloid is administered during sexual activity.

### FURTHER NUMBERED EMBODIMENTS

Further numbered embodiments of the present disclosure are as follows:

### Embodiment 1

A method of treating a sexual dysfunction in a human male subject in need thereof comprising administering an effective amount of a mesembrine alkaloid or salt thereof to the subject in need thereof.

### Embodiment 2

A method of delaying ejaculation in a human male subject comprising administering an effective amount of a mesembrine alkaloid or salt thereof to the subject.

### Embodiment 3

A method of delaying orgasm in a human male subject comprising administering an effective amount of a mesembrine alkaloid or salt thereof to the subject.

### Embodiment 4

The method of Embodiment 2 or 3, wherein the subject suffers from premature ejaculation.

### Embodiment 5

The method of Embodiment 2 or 3, wherein the subject is a healthy male who does not suffer from premature ejaculation.

### Embodiment 6

A method of enhancing one or more aspects of sexual activity in a human male comprising administering a mesembrine alkaloid or salt thereof to the male in need thereof.

### Embodiment 7

The method of Embodiment 7, wherein the one or more aspects are selected from increased or enhanced control over climax, increased or enhanced control over orgasm, increased or enhanced perception of pleasure by the subject and/or the subject's partner, enhanced or improved sexual performance, delay of ejaculation, and delay of orgasm.

### Embodiment 8

The method of any one of Embodiments 1-7, wherein the mesembrine alkaloid is selected from mesembrine, mesembrenone, Δ⁷mesembrenone, 4′-O-demethylmesembranol, mesembrenol, 4′-O-demethylmesembrenol, mesembranol, 4′-O-demethylmesembrenone, sceletenone, N-demethly-N-formulmesembrenone, O-acetylmesembrenol, mesembrane, N-demethelymesembrenol, N-demethylmesembranol, hordenine, joubertiamine, dehydrojoubertiamine, dehydrojoubertiamine, joubertinamine, O-methyldehydrojoubertiamine, O-methyoubertiamine, O-methyldihydrojoubertiamine, 3′-methoxy-4′-O-methyoubertiamine, 3′-methoxy-4′O-methyljoubertiaminol, 4-(3,4-dimethoxyphenyl-4-[2-acetylmethylamino)ethyl]cyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine.

### Embodiment 9

The method of any one of Embodiments 1-7, wherein the mesembrine alkaloid is selected from mesembrine, mesembrenone, mesembrenol, and tortuosamine.

### Embodiment 10

The method of Embodiments 1-7, wherein the mesembrine alkaloid is a compound of Formula (I).

### Embodiment 11

The method of any one of Embodiments 1-10, wherein the mesembrine alkaloid or salt thereof is administered sublingually, nasally, orally, parenterally, buccally, rectally, topically, transdermally, transurethrally, or by intracavernosal injection.

### Embodiment 12

The method of any one of Embodiments 1-11, wherein the mesembrine alkaloid or salt thereof is formulated with one or more pharmaceutically acceptable excipients.

### Embodiment 13

The method of any one of Embodiments 1-12, wherein the mesembrine alkaloid or salt thereof is formulated in a dosage form selected from a liquid, a lozenge, a fast disintegrating tablet, a lyophilized preparation, a film, a spray, or a mucoadhesive.

### Embodiment 14

The method of any one of Embodiments 1-12, wherein the spray is an oral spray, a nasal spray, or a topical spray.

### Embodiment 15

The method of any one of Embodiments 1-14, wherein the mesembrine alkaloid or salt thereof is administered as a single daily dose.

### Embodiment 16

The method of any one of Embodiments 1-14, wherein the mesembrine alkaloid or salt thereof is administered as multiple daily doses.

### Embodiment 17

The method of Embodiment 16, wherein the mesembrine alkaloid or salt thereof is administered two, three, four, or five times daily.

### Embodiment 18

The method of Embodiment 16, wherein the mesembrine alkaloid or salt thereof is administered three times daily.

### Embodiment 19

The method of any one of Embodiments 15-18, wherein each dose comprises at least 0.001 mg of the mesembrine alkaloid or salt, thereof.

### Embodiment 18

The method of any one of Embodiments 15-18 wherein each dose comprises at least 0.01 mg of the mesembrine alkaloid or salt thereof.

### Embodiment 20

The method of any one of Embodiments 15-18, wherein each dose comprises between about 0.001 mg and about 500 mg of the mesembrine alkaloid or salt thereof.

### Embodiment 21

The method of any one of Embodiments 15-18, wherein each dose comprises between about 500 mg and about 1000 mg of the mesembrine alkaloid or salt thereof.

### Embodiment 22

The method of any one of Embodiments 1-21, wherein the mesembrine alkaloid or salt thereof is administered at least 10 minutes, at least 20 minutes, at least 30 minutes, at least one hour, at least 2 hours, at least 3 hours, at least 4 hours, or at least 5 hours prior to sexual activity.

### Embodiment 23

The method of any one of Embodiments 1-21, wherein the mesembrine alkaloid or salt thereof is administered about one hour prior to sexual activity.

### Embodiment 24

The method of any one of Embodiments 1-21, wherein the mesembrine alkaloid or salt thereof is administered about 20 minutes prior to sexual activity.

### Embodiment 25

The method of any one of Embodiments 1-24, wherein the sexual dysfunction is premature ejaculation.

### Embodiment 26

The method of Embodiment 25, wherein the administration of the mesembrine alkaloid or salt thereof prevents premature ejaculation.

### Embodiment 27

The method of Embodiment 25, wherein the administration of the mesembrine alkaloid or salt thereof extends the amount of time during sexual activity before ejaculation occurs.

### Embodiment 28

The method of any one of Embodiments 25-27, wherein the method does not result in a concurrent increase or onset of negative symptoms such as low sex drive or difficulty obtaining or maintaining an erection.

### Embodiment 29

The method of any one of Embodiments 1-28, wherein the mesembrine alkaloid is an extract from a botanical source.

### Embodiment 30

The method of Embodiment 29, wherein the extract is a substantially pure extract of the mesembrine alkaloid.

### Embodiment 31

The method of any one of Embodiments 1-28, wherein the mesembrine alkaloid is synthesized.

### Embodiment 32

The method of any one of Embodiments 1-31, wherein the mesembrine alkaloid is administered in combination with another drug product.

### EXAMPLES

### Example 1

### Alkaloid Extraction Methods

Mesembrine extraction methods are described in WO 1997/046234. The methods are briefly summarized as follows.

Dry material: Material (or alcoholic extracts) are air-dried at maximum 40° C. before analysis. Yield figures for mesembrine are variable but are typically between 15 and 35 mg per gram dry leaves (mean value around 15 mg per gram dry weight). Finely ground material (pestle and mortar) is mixed with 15 mL 0.05 M H₂SO₄ and left standing at room temperature for 20 minutes. After filtration, the remaining solids are re-extracted with 5 mL 0.05 M H₂SO₄. The aqueous phases are combined, applied to glass columns with a coarse grade celite (24 g), alkalinized with ammonia (4 mL) and extracted (1×) with 100 mL CH₂Cl₂. The CH₂Cl₂ extracts are dried with anhydrous Na₂SO₄ and the solvent is evaporated under reduced pressure to leave the alkaloid as a pale brown oil. The alkaloids can also be extracted with hot or cold water instead of H₂SO₄, or with methanol, ethanol, acetonitrile, chloroform, or dichloromethane.

| | Δ7- | | | | Sceletium- | Total |
|---|---|---|---|---|---|---|
| | mesembrenone | mesembrenol | mesembrenone | mesembrine | A4 | alkaloids |
| % | 0.07 | 0.04 | 0.71 | 3.72 | 0.08 | 4.62 |

Fresh material: The leaf sap of fresh leaves (or alcoholic extracts) can be studied directly, or the alkaloids may be directly extracted in hot or cold water, ethanol, ethanol/acetonitrile, chloroform, or dichloromethane or any other suitable solvent. For HPLC or GC, the sample has to be filtered (e.g., 0.45 μm filter) in order to protect the columns from impurities. Yield figures for mesembrine are variable but are typically between 0.8 and 6.5 mg per mL leaf sap (mean value around 3.3 mg/mL).

### Example 2

### Mesembrine Extract Administration Increases Ejaculatory Latency Time in Human Males

A composition of dry extracted mesembrine alkaloid is administered sublingually to determine the effect on increased ejaculatory latency time in human male subjects 100 mg of extracted dry material containing approximately 4.62% mesembrine alkaloids was administered sublingually to human male subjects (n=3) 20 minutes prior to intravaginal sexual activity with a female partner. As a vehicle control (aka “Vehicle”), dried Aloe vera extract was provided. Latency time of ejaculation was measured and calculated by time from start of sexual activity to ejaculation. Results are provided Table 1 below. In all test cases, delay in ejaculation coincided with delay in orgasm.

TABLE 1
Ejaculation latency results

| | Vehicle | 100 mg dry extract |
|---|---|---|
| Ejaculatory Latency (sec) | 188 ± 45 s | 923 ± 128 s |

### Example 3

### Effect of Mesembrine and Mesembrenone Alkaloids in a Rat Para-Chloroamphetamine (PCA)-Induced Ejaculation Model

Experiments were performed to assess the effect of an acute treatment of alkaloids, mesembrine, mesembrenone, mesembrenol and mesembranol, at their maximal tolerable dose (MTD) on ejaculation latency in a para-chloroamphetamine (PCA)-induced ejaculation model in anaesthetized Wistar rats.

Adult male Wistar rats (Charles-River, France, 10-11 weeks old) were housed at least 10 days prior to the beginning of the experiments with free access to regular diet and water and maintained on an inversed 12 hour dark/light cycle (9:00/21:00). All procedures were performed in accordance with the legislation on the use of laboratory animals (NIH publication N°85-23, revised 1996) and Animal Care Regulations in force in France as of 1988 (authorization from competent French Ministry of Agriculture—Agreement No. B78-423-1, July 2017).

At 12 weeks of age, the rats were weighed and randomized into study groups in a way to have a comparable mean body weight between study groups. Rats received a single administration by intraperitoneal (IP) route of either vehicle, mesembrine, mesembrenone, mesembrenol, or mesembranol 1 hour before the in vivo evaluation of PCA-induced ejaculation as follows:

Part 1: Rats received a single IP injection of either vehicle control (0.5% hydroxypropyl methylcellulose (HPMC) in sterile water, n=10), mesembrine at 20 mg/kg (n=9), 10 mg/kg (n=11), 2 mg/kg (n=12), 200 μg/kg (n=12) and 20 μg/kg (n=10) 1 hour prior to administration of PCA. Treatment groups for Part I are outlined below in Table 2:

TABLE 2
Experimental Groups for Part 1

| Drug | Dose | Treatment administration: | N |
|---|---|---|---|
| Vehicle | — | Single IP administration delivered | 10 |
| Mesembrine | 20 mg/kg (MTD) | 1 h before PCA administration | 9 |
| Mesembrine | 10 mg/kg | | 11 |
| Mesembrine | 2 mg/kg | | 12 |
| Mesembrine | 200 μg/kg | | 12 |
| Mesembrine | 20 μg/kg | | 10 |

Part 2: Rats received a single IP injection of mesembrenone at 20 mg/kg (n=10). Treatment groups for Part 2 are outlined in Table 3.

TABLE 3
Experimental Groups for Part 2

| Drug | Dose | Treatment administration: | N |
|---|---|---|---|
| Mesembrenone | 20 mg/kg | Single IP administration delivered | 10 |
| | | 1 h before PCA administration | |

Part 3: Rats received single IP injection of mesembrenol at 20 mg/kg (n=4) or 10 mg/kg (n=6) or mesembranol at 20 mg/kg (n==6) or 10 mg/kg (n=6). Treatment for Part 3 are outlined in Table 4.

TABLE 4
Experimental Groups for Part 3

| Drug | Dose | Treatment administration: | N |
|---|---|---|---|
| Mesembrenol | 20 mg/kg | Single IP administration delivered 1h | 4 |
| Mesembrenol | 10 mg/kg | before PCA administration | 6 |
| Mesembranol | 20 mg/kg | | 6 |
| Mesembranol | 10 mg/kg | | 6 |

After treatment administration, the rats underwent an evaluation of their ejaculation function induced by PCA administration. Thus, the expulsion of seminal plug(s) and seminal vesicle pressure (SVP) increase was evaluated. At the end of experimental period, the rats were euthanized by an intracardiac injection of pentobarbital (Eutasol®, Centravet, France).

### Methods

Drug Preparation. Alkaloids were diluted to a 100 mg/mL final concentration (stock solution) and mixed by vortex. Then, aliquots per rat were prepared and stored at −20° C. until use. The day of experiment, 1 aliquot per rat was thawed and diluted to the desired concentration. The volume of administration was either 2 mL/kg for the IP administration or 5 mL/kg for the oral gavage, in line with good practice for administration volume in rat.

Ejaculation was induced by intraperitoneal administration of P-chloroamphetamine (PCA) at 5 mg/kg in anesthetized rats according to previously described methods (Clement el al., Role of peripheral innervation in p-chloroamphetamine-induced ejaculation in anesthetized rats. J Androl 2006; 27:381-389). PCA is an amphetamine derivative that liberates catecholamines and serotonin from monoaminergic nerve terminals. Systemic administration of PCA has been reported to induce ejaculation in both conscious (Humphries et al, 1981; Rènyi, 1985) and anesthetized (Yonezawa et al, 2000) rats.

Seminal vesicle pressure (SW) was measured as a physiological marker for the emission phase of ejaculation (Clement P, Giuliano F. Physiology and Pharmacology of Ejaculation. Basic Clint Pharmacol Toxicol 201.6 119 Suppl 3:18-25),

Rats were anesthetized and their temperature maintained at 37° C. using a homoeothermic blanket. The carotid artery was catheterized with polyethylene tubings filled with heparinized saline (50 UI/ml) to record continuously blood pressure (BP) via a pressure transducer (Elcomatic 750, Glasgow, UK). Pressure was recorded using specific data acquisition software (Elphy, CNRS, France).

For measuring SVP, a suprapubic midline incision was performed to expose seminal vesicles and a polyethylene catheter (0.50 mm), filled with mineral oil, was inserted into one seminal vesicle through the apex and connected to a pressure transducer (Elcomatic 750, Glasgow, UK). The SVP was recorded and analyzed using specific data acquisition software (Elphy, CNRS, France).

Each recording session started 50 min after IP administration of vehicle or drug with 10 min baseline recording period followed by IP injection of PCA at 5 mg/kg. Thus, in vivo evaluation started 1 h after treatment administration.

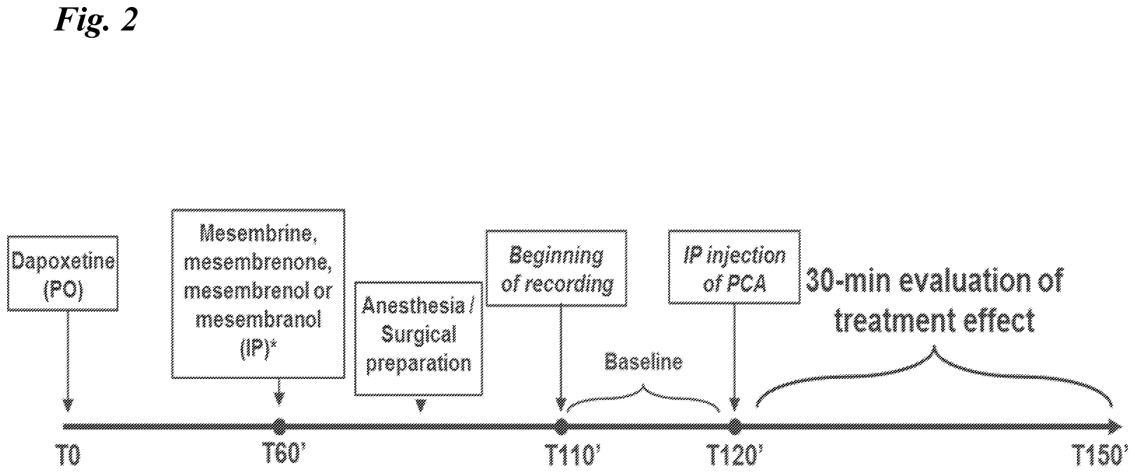

Seminal plug(s) expulsion and SVP increase were monitored over a 30-min period after PCA administration. An overview of the experimental protocol is provided in FIG. 2.

### Experimental Endpoints

The proportion of ejaculating rats (i.e., expulsion of a seminal plug associated with SVP rises) following PCA injection was determined. Then the following endpoints were determined:
(a) Primary endpoints: Latency of the first ejaculation (i.e., expulsion of a seminal plug, associated with SVP rise) was assessed following PCA administration.
(b) Secondary endpoints: Change in SVP was determined.

Of note, rats exhibiting a diastolic arterial pressure under 40 mmHg during baseline period were discarded from the study.

### Statistical Analyses

All results are presented as mean±SEM. The following statistical tests were used where appropriate:
(a) Parametric tests: Student's t-test when comparing 2 groups and multiple comparisons test following one-way ANOVA to compare 3 or more groups.
(b) Non-parametric tests: Mann-Whitney test when comparing 2 groups and multiple comparisons test following Kruskall-Wallis test when comparing 3 or more groups.

Statistical analyses were performed with GraphPad Prism® 6.05 software. p values <0.05 were considered significant.

### Part 1: Results

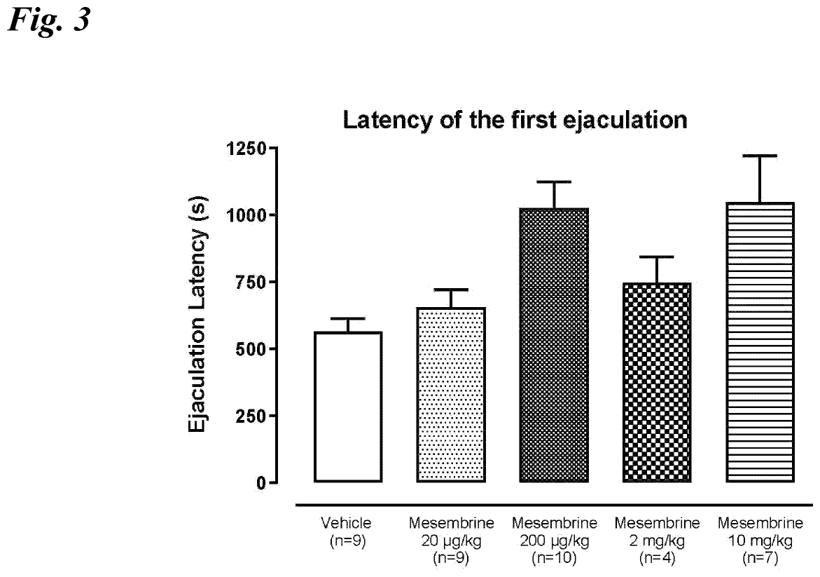

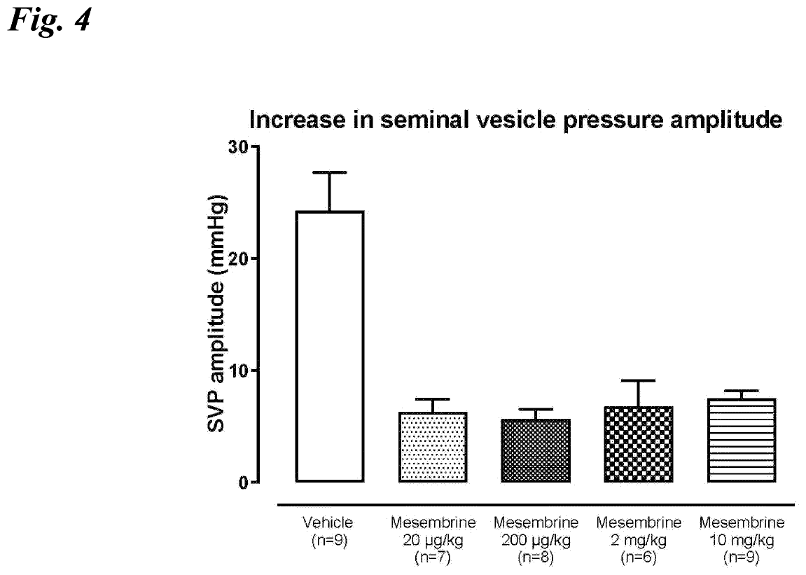

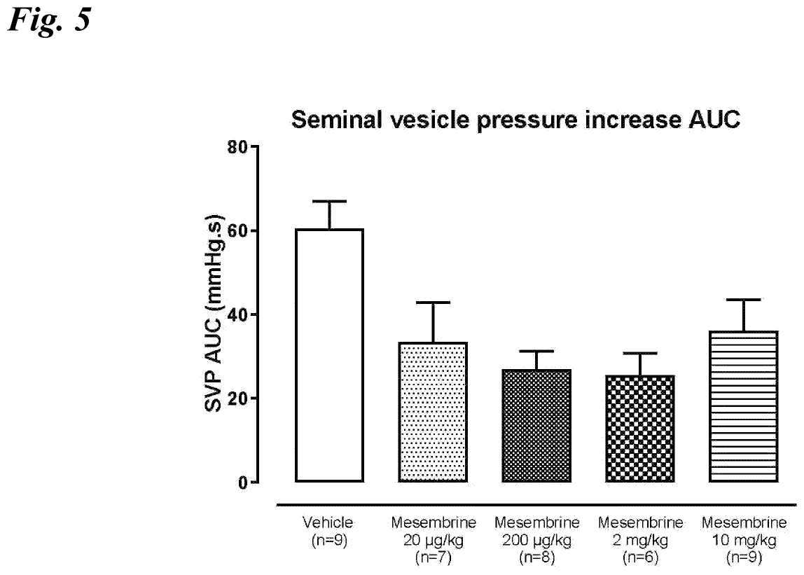

The results of Part 1 are shown in Table 5, FIG. 3, FIG. 4, and FIG. 5. As shown in Table 5, 100% of rats that received mesembrine at 20 mg/kg did not ejaculate during the 30-min observation period following PCA injection. In contrast, only 10% of rats receiving the vehicle control did not ejaculate during the observation period.

TABLE 5
Part 1 Ejaculation Results - Proportion of ejaculating rats

| | N of ejaculating | N of rats not | Percentage of |
|---|---|---|---|
| Groups | rats | ejaculating | ejaculating rats |
| Vehicle | 9/10 | 1/10 | 90% |
| Mesembrine 20 μg/kg | 9/9 | 0/9 | 100% |
| Mesembrine 200 μg/kg | 10/11 | 1/11 | 91% |
| Mesembrine 2 mg/kg | 4/12 | 8/12 | 33% |
| Mesembrine 10 mg/kg | 7/12 | 5/12 | 58% |
| Mesembrine 20 mg/kg | 0/10 | 10/10 | 0% |

Ejaculation latency is shown in FIG. 3. In all dosage conditions tested, treatment with mesembrine delayed ejaculatory response when compared to the vehicle control. Seminal vesicle pressure amplitude is shown in FIG. 4. Seminal vesicle pressure area under curve (AUC) is shown in FIG. 5. Seminal vesicle pressure response assessed as amplitude and AUC was decreased in all mesembrine treatment cases compared to vehicle control.

### Part 2: Results

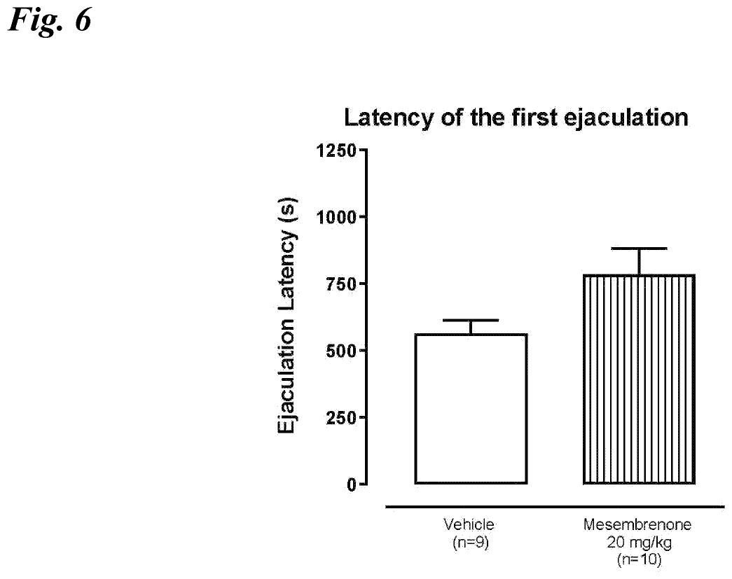

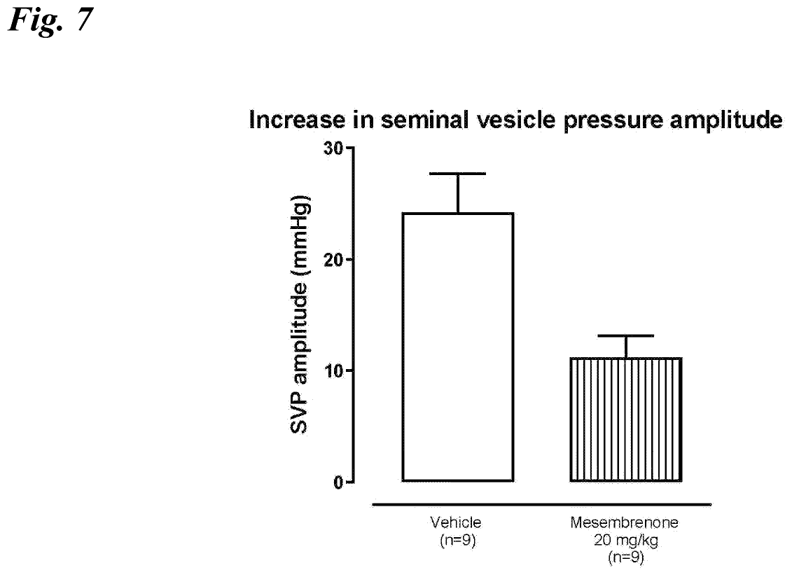

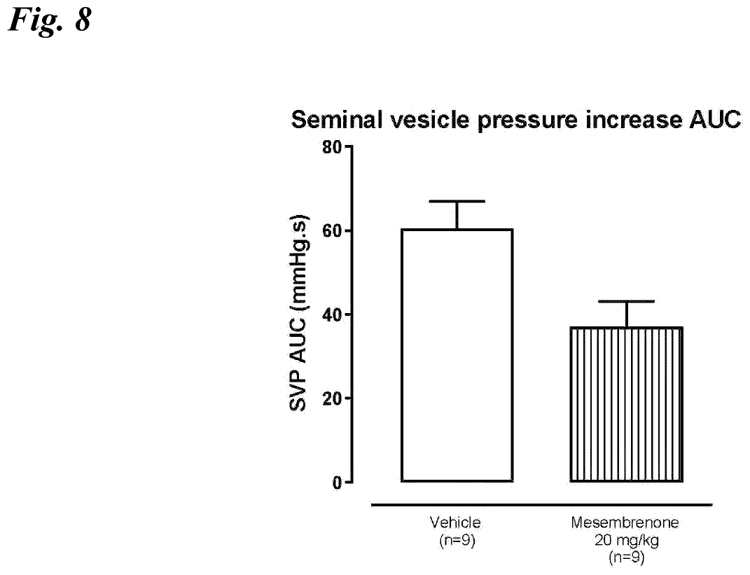

Ejaculation latency results of Part 2 are provided in FIG. 6. As shown in FIG. 6, rats that received mesembrenone at 20 mg/kg show delayed ejaculation during the 30-min observation period following PCA injection. Seminal vesicle pressure amplitude is shown in FIG. 7. Seminal vesicle pressure area under curve (AUC) is shown in FIG. 8. Seminal vesicle pressure response was decreased by mesembrenone treatment compared to vehicle.

### Part 3: Results

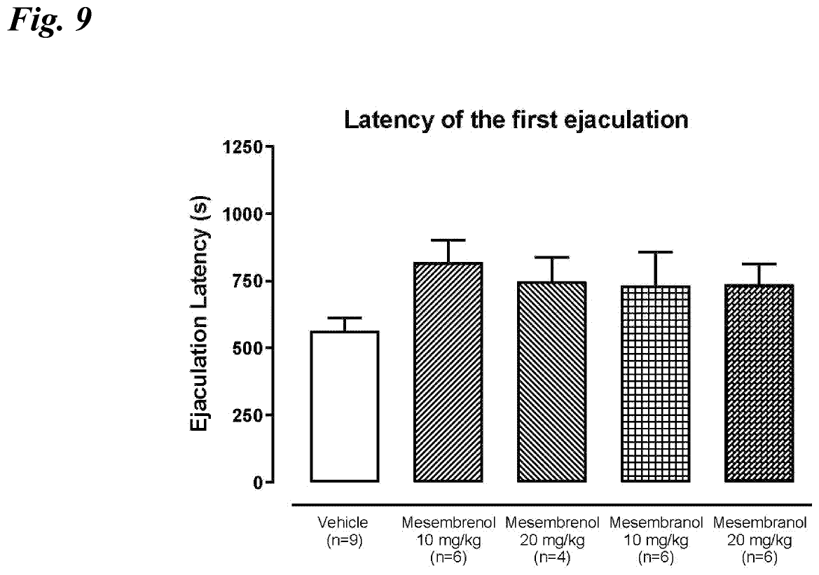

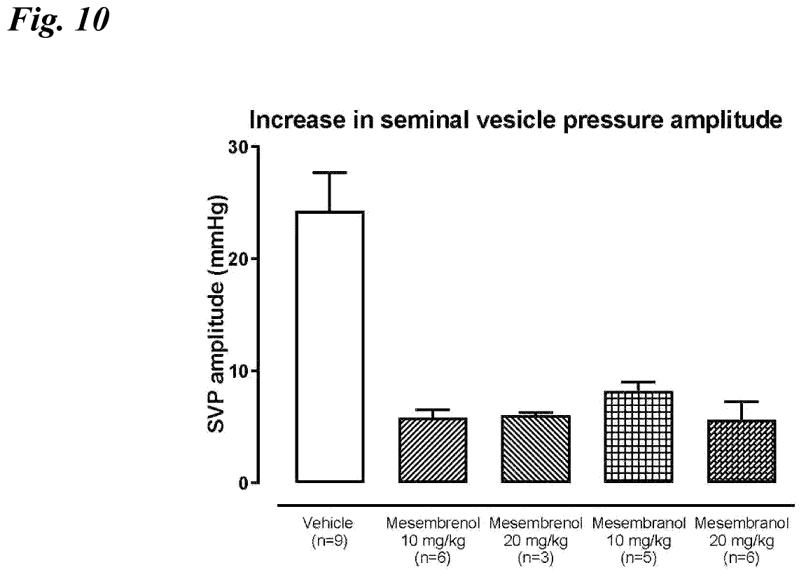

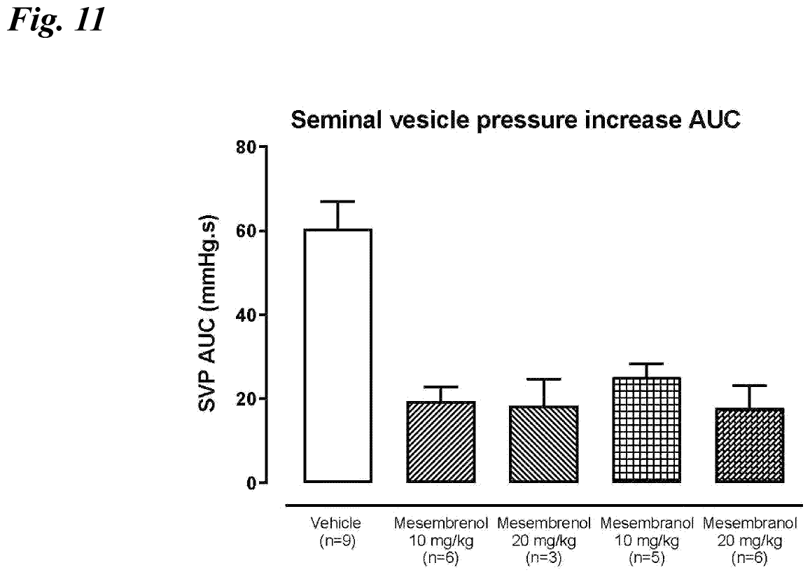

Ejaculation latency results of Part 3 are provided in FIG. 9. As shown in FIG. 9, rats that received mesembrenol and mesembranol show delayed ejaculation during the 30-min observation period following PCA injection. Seminal vesicle pressure amplitude is shown in FIG. 10. Seminal vesicle pressure area under curve (AUC) is shown in FIG. 11. Seminal vesicle pressure response was decreased by mesembrenol treatment compared to vehicle. Seminal vesicle pressure response was decreased by mesembranol treatment compared to vehicle.

### SUMMARY

In sum, the results show that all mesembrine-treated rats (20 mg/kg) did not ejaculate during the 30-minute observation period (vs. 10% of non-ejaculating rats in vehicle group). The results showed that decreasing the mesembrine dose allowed ejaculation during the 30-minute observation period. Further, mesembrenone, mesembrenol and mesembranol delayed ejaculation in rats compared to the vehicle control group. Seminal vesicle pressure, a physiological marker for ejaculatory response, was also decreased by treatment with mesembrine alkaloids (mesembrine, mesembrenone, mesembrenol and mesembranol) compared to vehicle control.

### Example 4

Delay of Ejaculation in Human Males by Administration of Mesembrine

Experiments were performed to determine the effects of pure mesembrine on ejaculation in male subjects. 0.5 mg, 1 mg, 2.5 mg, and 5 mg doses of pure mesembrine (Cayman Chemical Company, MI, USA) were administered by oral, nasal or sublingual routes to healthy human male subjects. The formulation for oral (swallowed) and sublingual (held under tongue for 5 minutes) was 2 mg mesembrine/mL 40% aqueous for oral and sublingual. The nasal spray formulation was 0.4 mg mesembrine/mL 1% w/w NaCl saline delivered via metered nasal spray device. Mesembrine was administered 20 minutes prior to sexual activity. The sexual activity was either solo masturbation (“self”) or partnered intercourse with a female partner (“partnered”). Latency time of ejaculation was measured and calculated by time from start of sexual activity to ejaculation. Results are shown below in Table 6. As shown, mesembrine extended the ejaculation latency at all doses tested. In all test cases, ejaculation coincided with orgasm. In all cases where ejaculation was not achieved, orgasm was also not achieved.

TABLE 6
Results of various mesembrine doses on ejaculation latency

| | Weight | Dose (mg | | | Latency | |
|---|---|---|---|---|---|---|
| Subject | (kg) | mesembrine) | Self/Partnered | Administration | time (min, sec) | Qualitative experience |
| A | 77 kg | 0.5 | Self | sublingual | 14:40 | Orgasm with drug was more intense than average |
| | | | | (5 min under | | masturbation orgasm without drug. Intensity and |
| | | | | tongue) | | perspiration level with drug akin to partnered |
| | | | | | | intercourse after several days/weeks without |
| | | | | | | intercourse or partnered intercourse (without |
| | | | | | | drug). |
| A | 77 kg | 1 | Self | sublingual | 18:02 | Onset after dosing with drug felt at around 9 min; |
| | | | | (5 min under | | subtle effect noted at 10 min, and noticeably |
| | | | | tongue) | | stronger at 1 mg than 0.5 mg |
| | | | | | | Felt body-wide relaxation/gentle stimulation as of |
| | | | | | | 15 min and until >25 min |
| A | 77 kg | 0 | Self | no drug | 2:41 | |
| B | 75 kg | 0 | Self | no drug | 3:31 | |
| B | 75 kg | 0.5 | Self | oral | 17:00 | Significant lengthening of latency time noticed |
| | | | | | | with drug. |
| C | 100 kg | 0 | Self | oral | 4:51 | |
| C | 100 kg | 0.5 | Self | oral | did not | Did not complete ejaculation as an effect of drug. |
| | | | | | complete | |
| D | 70 kg | 0 | Self | no drug | 3:23 | n/a |
| D | 70 kg | 0 | Partnered | no drug | 4:30 | n/a |
| D | 70 kg | 0.05 | Self | nasal spray (in | 18:38 | Effects noticed more rapidly than oral dosing. |
| | | | | aqueous 1% | | |
| | | | | w/w NaCl) | | |
| D | 70 kg | 1 | Self | oral | 10:42 | Satisfactory dosing to maintain control and easily |
| | | | | | | reach orgasm. |
| | | | | | | Orgasm notably more pleasurable with drug than |
| | | | | | | without drug |
| D | 70 kg | 2.5 | Self | oral spray | 24:32 | Onset of action with oral spray was faster than |
| | | | | | | oral dosing |
| D | 70 kg | 2.5 | Partnered | oral | 29:49 | Increased satisfaction from female partner noted. |
| D | 70 kg | 5 | Partnered | oral | did not | Did not complete ejaculation as an effect of drug. |
| | | | | | complete | |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1.-29. (canceled)

30. A method of treating sexual dysfunction in a human male subject, comprising administering an effective amount of mesembrine or a salt thereof.

31. The method of claim 30, wherein the subject suffers from premature ejaculation.

32. The method of claim 30, wherein the mesembrine or a salt thereof is administered sublingually, nasally, orally, parenterally, buccally, rectally, topically, transdermally, transurethrally, or by intracavemosal injection.

33. The method of claim 30, wherein the mesembrine or a salt thereof is formulated with one or more pharmaceutically acceptable excipients.

34. The method of claim 30, wherein the mesembrine or a salt thereof is formulated in a dosage form selected from a liquid, a lozenge, a fast disintegrating tablet, a lyophilized preparation, a film, a spray, or a mucoadhesive.

35. The method of claim 30, wherein the mesembrine or a salt thereof is administered as a single daily dose.

36. The method of claim 30, wherein the mesembrine or a salt thereof is administered as multiple daily doses.

37. The method of claim 30, wherein each dose comprises at least 0.001 mg of the mesembrine or a salt thereof.

38. The method of claim 30, wherein the mesembrine or a salt thereof is administered at least 10 minutes, at least 20 minutes, at least 30 minutes, at least one hour, at least 2 hours, at least 3 hours, at least 4 hours, or at least 5 hours prior to sexual activity.

39. The method of claim 30, wherein the administration of the mesembrine or a salt thereof prevents premature ejaculation.

40. The method of claim 30, wherein the mesembrine is produced from an extract from a botanical source.

41. The method of claim 40, wherein the extract is a substantially pure extract of mesembrine.

42. The method of claim 30, wherein the mesembrine is synthesized.

43. The method of claim 30, wherein the mesembrine is administered in combination with another drug product.
